(19) United States
(12) Patent Application Publication
(10) Pub. No.: US 2012/0067350 A1
(43) Pub. Date: Mar. 22, 2012

(54) Nasal Soft CPAP Cushion

(76) Inventor: Melvin Palmer, JR., Alexander City, AL (US)

(21) Appl. No.: 12/884,352

(22) Filed: Sep. 17, 2010

(51) Int. Cl. A61M 16/06

(52) U.S. Cl. 128/207.13

(57) ABSTRACT

A cushion to reduce prevent nasal skin breakdown and bridge soreness while preventing Continuous Positive Airway Pressure mask leaks is made from a tacky polymeric gel material and uses a unique shape overlying and conforming to the bridge of the nose and cheekbones to provide a comfortable soft seal between the mask and the skin of the wearer.

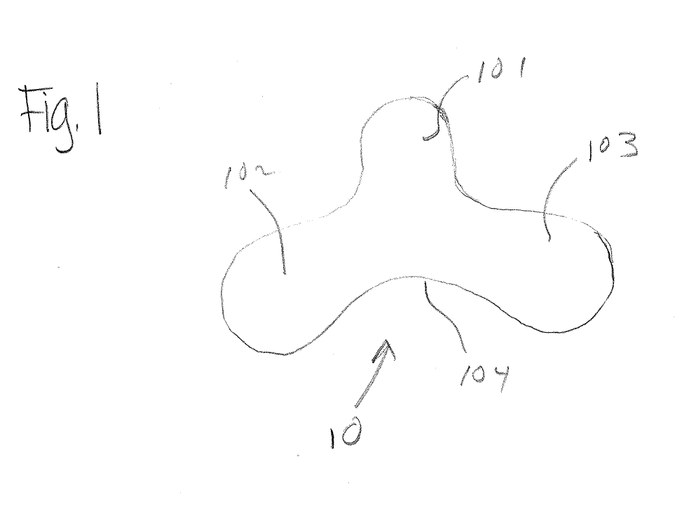

### BACKGROUND OF THE INVENTION

The present invention relates to the field of treatment for obstructive sleep apnea and more particularly to such treatment with Continuous Positive Airway Pressure (CPAP) with a CPAP machine which provides air at a constant prescribed pressure through a tube and out of a CPAP mask to a person with obstructive sleep apnea. Most CPAP masks in use today rest directly on the face on the bride of the nose and the soft tissue of the face. The pressure points and contact lead to nasal skin breakdown and bridge soreness. The variation in facial structure leads to mask leaks.

### SUMMARY OF THE PRESENT INVENTION

It is an object of the present invention to prevent nasal skin breakdown and bridge soreness while preventing mask leaks by providing a soft gel like barrier between the skin and the CPAP mask.

These and other objects and advantages of the invention will become apparent from the following detailed description of the preferred embodiment of the invention.

### BRIEF DESCRIPTION OF THE DRAWINGS

A cushion for a CPAP mask is depicted in the accompanying drawings which form a portion of this disclosure and wherein:

The FIGURE is a plan view of the cushion;

### DESCRIPTION OF THE PREFERRED EMBODIMENT

Referring to the FIGURE for a clearer understanding of the invention, it may be seen that the preferred embodiment of the invention contemplates a gel cushion 10 designed to overlie the bridge of a patients nose and the cheekbones. Nasal lobe 101 is intended to be centered on the bridge of the nose and wings 102 and 103 are symmetrically positioned beneath the eyes in the cheekbone area of the face. Subsequent placement of a CPAP mask of either the nasal or full face type will result in the pressure point of the mask will rest on the nasal lobe 101 of the cushion between the eyes. The wings 102 and 103 allow the mask to seal against the face with no leaks.

Cushion 10 is made from a gel that is heated and injected into a mold then allowed to cool and harden to the gel state. By gel, I mean a soft polymeric material made from synthetic rubber or an elastomeric material. Elastomers may include but are not limited to chlorinated polyethylene (CPE), ethylene polysulfide (ET), ethylene-propylene copolymers ((EPM); organopolysiloxane (SI), polybutadiene (BP), polyisoprene, or polyurethane (PUR).

The polymeric material may make up a gelatinous elastomeric composition that is a mixture of a thermoplastic elastomer and an oil (or plasticizer). In one embodiment of the present invention, the composition may be a mixture comprised of a styrenic block copolymer and anoil, where the oil is in excess, by weight, of the copolymer. In another embodiment, the composition may be a mixture comprised of a mixture of one or more styrenic block copolymers and at least two oils. In still another embodiment, the elastomeric material comprises a styrenic block copolymer and an oil mixture of a first oil and a second oil where the first oil has a viscosity that is greater than a viscosity of the second oil and the second oil is used in excess of the first oil.

These gelatinous elastomeric compositions are elastic, tear resistant, and soft even when damaged. They may be able to be stretched several times their original length. Cushion 10 will be a manufactured with a Shore A-DO hardness of 6 on a durometer scale, plus or minus 2.

The styrenic block copolymers, which are thermoplastic elastomers, have a structure normally consisting of a block of a rigid styrene on each end with a rubbery phase in the center. Styrenic block copolymers include, but are not limited to, SBS (styrene butadiene styrene), SIS (styrene isoprene styrene), SEPS (styrene ethylene/propylene styrene), SEBS (styrene ethylene/butylene styrene), and SEEPS (styrene ethylene/ethylene propylene styrene). These materials are commercially available from, for example, Kraton Polymers (Houston, Texas). Exemplary Kraton (US) products include KRATON 1651 (SEBS). It is possible to use just one of these polymers or a combination of these polymers.

Oil or plasticizer generally refers to mineral oil or silicone (dimethyl silaxone) oil. The oil is mixed with the styrenic block copolymer. The heavier oils (i.e., greater molecular weights or greater viscosity) decrease heat deformation and processibility and increase surface tack. Such oils are commercially available from, for example, Crompton Corporation (Witco Refined Products), Greenwich, Connecticut. Exemplary oils include: BLANDOL white mineral oil, specific gravity @ 250C/250C (ASTM D4052)-0.839/0.855; Kinematic viscosity @ 40 OC, CST (ASTM D445)-14.2/17.0 (heavy oil), and SEMTOL white mineral oil, specific gravity @ 250C/250C (ASTM D4052)-0.804/0.827, Kinematic viscosity @ 40 OC, CST (ASTM D445)-3.9/5.5 (light oil). It may also be possible to use natural oils such as corn oil, vegetable oil, olive oil, fish oil, or cod liver oil. Other conventional additives may also be added to the elastomeric material which include, but are not limited to, UV-stabilizer, heat-stabilizer, antimicrobial agents, antiviral agents, antifungus agents, antioxidants, pigments, glitters, dyes, or combinations thereof.

It will be known to those of ordinary skill in the art that by varying the amount of copolymer and oil one can achieve an end product having different durometer readings. The cushion may be injection molded at temperatures in excess of 300 degree F. in an aluminum mold that is temperature controlled by electric resistance heaters and cooling fluid from a recirculating system. The cushion has a uniform thickness of between about 0.125 inches and 0.250 inches.

Preferably, the cushion 10 will be tacky to the touch such that it is easier to obtain a good seal with a mask. Anatomically, the cushion nose lobe 101 integrates with the wings 102 and 103 to form a radiused curve that fits from the bridge of the nose to below the eye of the patient. The wings 101 and 102 join as the margin of the cushion crosses the nose above the nostrils and is curved at 104 to allow the cushion to lay flat against the skin over the entire cushioned area.

It is to be understood that the form of the invention shown is a preferred embodiment thereof and that various changes and modifications may be made therein without departing from the spirit of the invention or scope as defined in the following claims.

What is claimed is:

1. A cushion for use with a Continuous Positive Airway Pressure mask comprising:
a. a nose lobe adapted to extend along the nose of a wearer between the eyes of the wearer overlying the bridge of the nose and interposed between the nose of the wearer and a mask;
b. a pair of wings integrally formed with said nose lobe and extending from said nose lobe in a manner such that they wings may overlie the cheekbones of said wearer ;
wherein said cushion is made from a polymeric gel material having a Shore A-DO hardness of between 4 and 8 on a durometer scale.

2. A cushion as defined in claim 1 wherein said cushion has a uniform thickness of between about 0.125 inches and 0.250 inches.

3. A cushion as defined in claim 2 wherein said cushion is tacky to touch.

4. A cushion as defined in claim 3 wherein said cushion has an arcuate lower margin intermediate said wings, such that said cushion conforms to the facial features of the wearers face and provides a seal between said wearer's face and said CPAP mask.

5. A cushion as defined in claim 1 wherein said cushion has an arcuate lower margin intermediate said wings, such that said cushion conforms to the facial features of the wearers face and provides a seal between said wearer's face and said CPAP mask.

6. A cushion as defined in claim 5 wherein said cushion is tacky to touch.

7. A cushion as defined in claim 6 wherein said cushion has a uniform thickness of between about 0.125 inches and 0.250 inches.

8. A cushion as defined in claim 1 wherein said cushion is tacky to touch.

9. A cushion for use with a Continuous Positive Airway Pressure mask comprising:
a. a nose lobe adapted to extend along the nose of a wearer between the eyes of the wearer overlying the bridge of the nose and interposed between the nose of the wearer and a mask;
b. a pair of wings integrally formed with said nose lobe and extending from said nose lobe in a manner such that the wings may overlie the cheekbones of said wearer ;
wherein said cushion is made from a polymeric gel material having a uniform thickness of between about 0.125 inches and 0.250 inches and wherein said gel material allows said cushion to conform to the facial features of the wearers face and provide a seal between said wearer's face and said CPAP mask.

10. A cushion as defined in claim 9 wherein said cushion is tacky to touch.

11. A cushion as defined in claim 9 wherein said polymeric gel material has a Shore A-DO hardness of between 4 and 8 on a durometer scale.

12. A cushion as defined in claim 11 wherein said cushion is tacky to touch.

13. A cushion as defined in claim 12 wherein said cushion has an arcuate lower margin intermediate said wings.

14. A cushion as defined in claim 12 wherein said cushion nose lobe is integral with said pair of wings and is cooperatively positioned relative to said wings to form a radiused curve that fits from the bridge of the nose to below the eye of the wearer.
